(19) United States
(12) Patent Application Publication
(10) Pub. No.: US 2018/0338704 A1
(43) Pub. Date: Nov. 29, 2018

(54) ELASTIC BELT OR BAND WITH FERROMAGNETIC PROPERTIES (AND ANTIMICROBIAL PROPERTIES) FOR CLEAN AND SIMPLE PLACEMENT OF A MEDICAL MONITORING DEVICE USING MAGNETS

(71) Applicant: KONINKLIJKE PHILIPS N.V., EINDHOVEN (NL)

(72) Inventors: Ernest LAMAN, WAARLE (NL); Eefje Janet ARTS-HORNIX, NUENEN (NL); Lotte MEDEMA, GRONINGEN (NL); Pavan DADLANI, EINDHOVEN (NL)

(21) Appl. No.: 15/553,605

(22) PCT Filed: Mar. 16, 2016
(86) PCT No.: PCT/IB2016/051467

§ 371 Date: Aug. 25, 2017

Related U.S. Application Data
(60) Provisional application No. 62/138,481, filed on Mar. 26, 2015.

(51) Int. Cl. A61B 5/083; A41F 19/00

(52) U.S. Cl. CPC A61B 5/083; A41F 19/00

(57) ABSTRACT

A band (10) for positioning a medical monitoring device (14) on a portion of a target portion of a medical subject is provided. The band (10) includes a flexible main band body (12) that includes at least one of at least one plastic layer (28), a textile layer, and a flexible rubber layer. At least one ferromagnetic portion (30) is disposed on or in at least a first end portion (22) of the flexible main band body (12). At least one magnet (26) is disposed on or in a second end portion (24) of the flexible main band body opposite from the first end portion (22). The flexible main band body (12) is sized to wrap around the target portion of the medical subject with the first and second end portions (22, 24) overlapping such that the at least one magnet (26) and the at least one ferromagnetic portion (30) magnetically couple to secure the flexible main band body (12) wrapped around the target portion of the medical subject.

### FIELD

The following relates generally to medical monitoring devices, medical monitoring device placement systems, and related arts. However, it is to be understood that it also finds application in other usage scenarios and is not necessarily limited to the aforementioned application.

### BACKGROUND

In developing countries, pneumonia and other respiratory illnesses are prevalent, and have high mortality rate, especially for children under five years old. Rapid and accurate assessment of respiration, especially respiration rate, is a key diagnostic tool for assessing these illnesses. Conventionally, a hockey puck-sized accelerometer-based respiration monitor is attached to the chest with adhesive, either directly or using a disposable pouch. If it is directly attached, the device must be sterilized between patients. However, in developing countries, both the supply of disposable pouches, and the availability of sterilization equipment, may be limited.

Another issue with existing respiration monitor practice is that the monitor often needs to be re-positioned if its initial placement causes discomfort to the patient or does not provide a sufficient respiration signal. This is particularly an issue with infants and young children. Detaching and re-attaching the respiration monitor using an adhesive is problematic.

The following provides new and improved methods and systems which overcome the above-referenced problems and others.

### BRIEF SUMMARY

It is recognized that known systems and methods of attaching a respiration monitor to a patient, especially a child, has deficiencies. For example, the respiration monitor should be placed in a disposable pouch, or if directly attached to the patient should be sterilized between patients. In developing counties, however, the supply of disposable pouches and the availability of sterilization equipment may be limited. In addition, the monitor often needs to be re-positioned if its initial placement causes discomfort to the patient or does not provide a sufficient respiration signal.

Various improvements are disclosed herein.

In some illustrative embodiments, an elastic magnetic belt or band is provided for placement of a medical monitoring device (e.g., an accelerometer-based respiration monitor). The band is made of polyurethane or another flexible, elastic, and easily sanitized material. A magnet at one end of the band serves as the “buckle.” The remainder of the belt (or at least a length of the belt distal from the buckle end) has embedded or dispersed ferromagnetic material effective to magnetically attach the magnet buckle. After positioning the belt around the patient's chest, the respiration monitoring device is attached to the belt either using an adhesive or, in preferred embodiments, using a magnet on a back side of the monitoring device.

The approach has numerous advantages. It provides a “one size fits all” solution (for smaller patients, there is merely a belt “tail” left over). If the magnetic connection strength is suitably chosen, the belt provides self-adjusting tightness. For example, it can initially be wrapped around the chest slightly over-tight, and slippage of the magnet buckle provides loosening to the desired tightness. As a result, re-positioning of the belt and/or monitor is straightforward, especially if the monitor is magnetically mounted to the belt.

The belt can be advantageously easily sterilized (where available), and does not include seams or other features that can trap contaminants and possibly serve as a cross-contamination conduit. To further reduce seams or other contaminant traps, the magnetic buckle may comprise magnetic particles dispersed into the belt at the buckle end to provide the magnet without any seams or connectors. The belt can be re-used from patient to patient. To further address the cross-contamination issue, an antimicrobial dispersant or coating can be added to the belt, such as dispersing zeolite in the belt material.

The belt is particularly well-suited for respiration monitors as they do not need to contact the patient's skin. However, the belt can be used for other types of vital sign sensors. For example, a heart rate/SpO₂ monitor can be magnetically attached to the belt and optically coupled with the skin via an aperture opening in the belt. The belt can also be sized and placed otherwise than on the patient's chest. For example, the belt can be configured as a headband, wristband, or the like. Another contemplated variant is to replace the embedded magnetic material with ferromagnetic rivets spaced apart along the length of the belt. Depending upon the rivet design, this approach could introduce seams; however, rivets have an advantage in providing a compact mass of ferromagnetic material to which the magnetic buckle can attach. The disclosed positioning/holding belt may be used for both children and adults (as it is readily made “one-size-fits-all”).

In accordance with one aspect, a band for positioning a medical monitoring device on a portion of a target portion of a medical subject is provided. The band includes a flexible main band body that includes at least one plastic layer. At least one ferromagnetic portion is disposed on or in at least a first end portion of the flexible main band body. At least one magnet is disposed on or in a second end portion of the flexible main band body opposite from the first end portion. The flexible main band body is sized to wrap around the target portion of the medical subject with the first and second end portions overlapping such that the at least one magnet and the at least one ferromagnetic portion magnetically couple to secure the flexible main band body wrapped around the target portion of the medical subject.

In accordance with another aspect, a belt (10) for positioning a medical monitoring device (14) on a target portion of a medical subject is provided. The belt includes a main belt body that includes a flexible and elastic material. At least one ferromagnetic portion is disposed on or in at least a first end of the main belt body. A buckle magnet is disposed on or in a second end of the main belt body opposite from the first end portion of the main belt body. The main belt body is sized to wrap around the target portion of the medical subject with the first and second ends overlapping such that the buckle magnet and the at least one ferromagnetic portion magnetically couple to buckle the belt around the target portion of the medical subject.

In accordance with another aspect, a respiratory monitoring method is provided. A belt is wrapped around the chest of a subject. The belt wrapped around the chest of a subject is magnetically buckled by magnetically coupling a buckle magnet disposed at one end of the belt with ferromagnetic material disposed in or on a portion of the wrapped belt overlapping the buckle magnet. Patient respiration is monitored using a respiratory monitor attached to the belt.

One advantage resides in a medical monitoring device belt wearable for users of any size.

Another advantage resides in a medical monitoring device belt that self-adjusts a tightness thereof around the user.

Another advantage resides in a sterilizable medical monitoring device belt with limited or eliminated seams or contaminant traps.

Still further advantages of the present disclosure will be appreciated to those of ordinary skill in the art upon reading and understanding the following detailed description.

### BRIEF DESCRIPTION OF THE DRAWINGS

The present disclosure may take form in various components and arrangements of components, and in various steps and arrangements of steps. The drawings are only for purposes of illustrating the preferred embodiments and are not to be construed as limiting the present disclosure.

FIG. 1 shows a medical monitoring device in communication with a band in one embodiment of the present disclosure.

FIG. 2 shows a perspective view of a portion of the band of FIG. 1.

FIG. 3 shows a band in another embodiment of the present disclosure.

FIG. 4 shows an example use of the band of FIG. 1.

### DETAILED DESCRIPTION

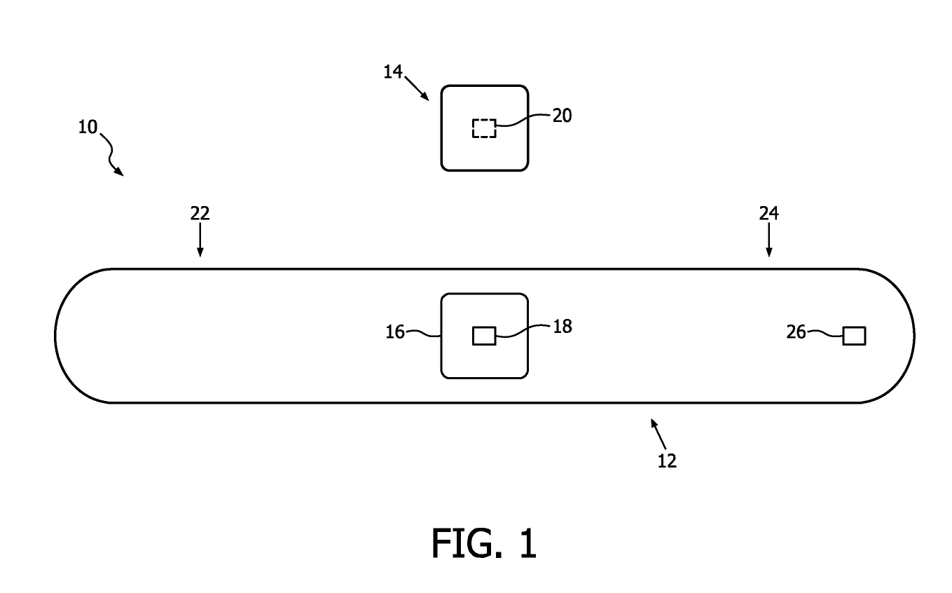

With reference to FIG. 1, a belt or band 10 for attachment to a patient is shown. As used herein, the term “belt” refers to a wearable device that can be wrapped around, and secured to, a target tissue of a patient. It will be appreciated that the terms “belt” and “band” can be used herein interchangeably, as described in the following. Moreover, as used herein, the term “target tissue” refers to any desired target tissue (e.g., tissue of a body part such as a chest, a waist, a wrist, an upper arm, a leg, a forehead, and the like) of a patient. As shown in FIG. 1, the band 10 includes a main band or belt body 12. The main band body 12 has a generally rectangular shape; although other shapes are possible (e.g., circular, triangular, square, and the like). A medical monitoring device 14 mounts to the main band body 12 at a device mounting location or portion 16. The monitoring device 14 may, for example, be a vital sign(s) monitoring device. In the illustrative embodiments, the monitoring device 14 monitors the respiration vital sign using an accelerometer-based respiration sensor. Additionally or alternatively, the monitoring device 14 may monitor other vital signs, such as cardiac pulse and/or peripheral blood oxygenation using an optical SpO₂ sensor. The main band body 12 is flexible enough for adjustment and patient comfort. The band 12 may be elastic, which has benefits in terms of comfort and providing a taut fit for respiration monitoring; however, an inelastic belt is also contemplated. As shown in FIG. 1, the main band body 12 is configured to receive the medical monitoring device 14 (e.g., a respiration monitoring device, a SpO₂ sensor, a heart rate monitor, and the like).

To do so, the main band body 12 includes the device-mounting portion 16 to receive and hold the medical monitoring device 14 on the main band body 12. The device-mounting portion 16 comprises a portion of the main belt body 12 which in illustrative FIG. 1 is delineated by an outline (e.g., a receiving recess) corresponding to a shape of the medical monitoring device 14. Optionally, the device-mounting portion 16 includes an aperture or an indentation (not shown) in the main belt body 12 for receiving a portion of the medical monitoring device 14. In a further example, the device-mounting portion 16 includes a protruding portion (not shown) for receiving a portion of the medical monitoring device 14. The medical monitoring device 14 is configured to be securely attached to the device-mounting portion 16. In one example, a magnet 18 disposed on the device-mounting portion 16 is configured for magnetic attraction to a device mounting magnet 20 disposed on the medical monitoring device 14. In another example, the medical monitoring device 14 is attached to the device-mounting portion 16 with an adhesive (not shown). In some embodiments, the main band body 12 does not include any defined device mounting portion 16, and the medical monitoring device 14 may be mounted anywhere along the length of the belt 12 using adhesive, a mounting magnet or so forth. As another contemplated variant, the main band body 12 does not include any defined device mounting portion, and the medical monitoring device 14 may be mounted anywhere along a substantial portion of the length of the belt 12, which substantial portion includes ferromagnetic material, using a mounting magnet. In further examples, the device-mounting portion 16 can be made from a breathable material so that superfluous humidity is transported away from the target portion of the medical subject.

The main band body 12 includes a first end portion 22 and an oppositely disposed second end portion 24. At least one magnet 26 is disposed on the second end portion 24. As shown, the magnet 26 is square; although other shapes are possible (e.g., circular, triangular, and the like). It will be appreciated that more than one magnet 26 can be disposed on the second end portion 24. The magnet 26 is configured to magnetically engage a portion of the first end portion 22 (that is, the magnet 26 serves as a magnetic “buckle” for the belt 12), as described in more detail below.

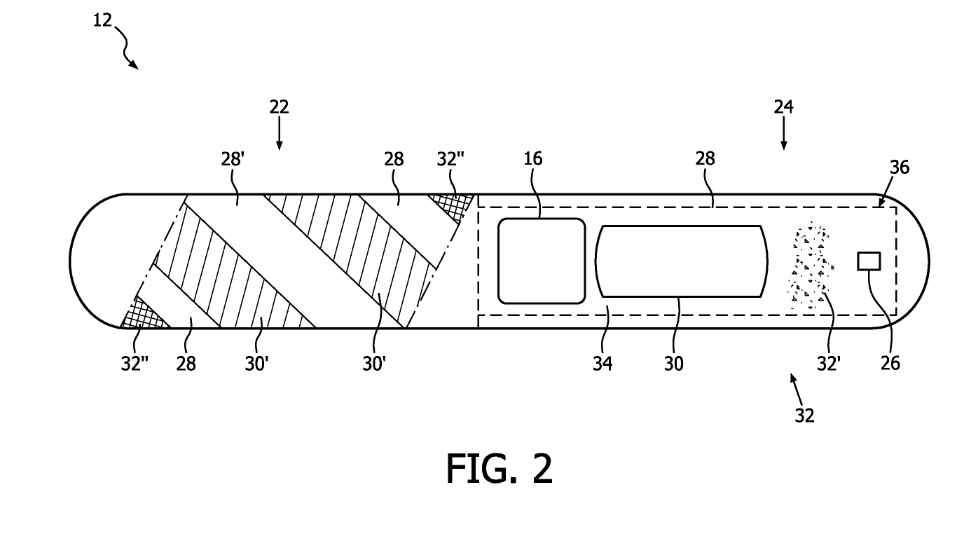

FIG. 2 depicts some illustrative contemplated compositions of the main band or belt body 12 so as to provide ferromagnetic material to which the magnetic buckle 26 engages. FIG. 2 also includes a cut-away portion to show an example interior embodiment of the main band body 12. Although FIG. 2 is depicted as showing multiple external and internal layers of the main belt body 12 within one embodiment of the band 10, it will be appreciated that any combination of exterior and interior layers of material (described in more detail below) can be used to manufacture the main belt body 12. As shown in FIG. 2, the main band body 12 is made of one or more layers of material. In some instances, the main band body 12 is made from at least one plastic layer 28 (polyethylene, polyurethane, polypropylene, and the like). For example, in one embodiment the main band body 12 is made entirely from one or more polyurethane layers 28′. In another example, the main band body 12 is made from one or more silicone layers (not shown). In further examples, the main band body 12 is made from a suitable elastic material (e.g., a textile layer, a flexible rubber layer, and the like). More generally, the band 12 is made of a non-magnetic material (although as described herein it has embedded magnetic material, e.g., embedded magnetic particles, secured rivets or the like).

In other embodiments, the main band body 12 includes at least one ferromagnetic portion 30 disposed on a portion thereof. For example, in one embodiment the main band body 12 includes at least one ferromagnetic layer 30′ embedded therein. In one embodiment, the ferromagnetic layer 30′ can rest above (or below) the plastic layer 28. In another embodiment, the ferromagnetic layer 30′ is embedded or mixed with the plastic layer 28. In a further embodiment, when the main band body 12, is made from silicone layers, the ferromagnetic portion 30 can include ferromagnetic particles makes the band 10 conductive, thereby making it non-static. As a result, dust collection is advantageously substantially reduced on the band 10. Advantageously, the ferromagnetic layer 30′ is configured to engage the magnet 26 (i.e., by magnetic attraction), thereby connecting the first and second end portion 22 and 24 to wrap the band 10 around the target tissue of the patient.

In general, one end 22 of the belt 10 includes a permanent magnet 26, while the opposite end 24 includes a ferromagnetic material 30 that is not magnetized extending along a length of that end at least sufficient to provide an amount of belt-fit adjustability to accommodate a contemplated range of chest sizes. In some instances, the at least one magnet 26 includes an electromagnetic portion. In some embodiments, the entire belt 10 is embedded with or otherwise includes magnetic material layer 30. The magnetic material layer 30 is preferably a ferromagnetic material such as iron, iron alloy, steel, nickel, nickel alloy, or so forth. The magnetic buckle 26 may be a permanent steel magnet, a ceramic or ferrite magnet, a rare-earth magnet (e.g., samarium-cobalt or neodymium-iron-boron magnet), or so forth. The magnet 26 is suitably chosen based on factors such as cost and desired magnetic coupling strength. For example, steel magnets tend to be low cost, while rare earth magnets are more expensive but tend to be stronger.

The strength of the magnetic coupling between the magnetic buckle 26 and the ferromagnetic material 30, 30′, 30″ is determined by factors such as the strength of the magnet forming the magnetic buckle 26, the density of the ferromagnetic material 30, 30′, 30″, and the extent of intervening non-magnetic material (if any) between the magnet 26 and the ferromagnetic material 30, 30′, 30″. A direct contact between the magnet 26 and the ferromagnetic material 30, 30′, 30″ provides the strongest coupling, but it may be advantageous to embed the ferromagnetic material 30, 30′, 30″ and/or the magnet 26 inside the material of the main belt body 12 in order to facilitate sterilization and avoid contaminant traps on the surface of the main belt body 12, which leads to some intervening plastic or other intervening non-magnetic material. Having some non-magnetic intervening material can beneficially enhance slippage of the coupling (e.g., reduce the coupling strength and/or reduce the static friction at the coupling) to enable the self-adjusting tightness feature of certain embodiments as described elsewhere herein. In instances where the magnet 26 includes an electromagnetic portion, the ferromagnetic portion 30 has slippage controlled by adjusting a voltage applied to the flexible main band body 12 to release any over-tightness of the wrapping of the flexible main band body 12 around the target portion of the medical subject. For example, the electromagnet 26 includes a release functionality based on the medical monitoring accelerometer device 14 and an emergency release algorithm input (not shown).

Advantageously, by embedding the ferromagnetic layer 30 within the plastic layer 28, the main band body 12 is free of seams, thereby preventing contamination of the band or belt 10.

In another aspect, the main belt body 12 can include a sterilizable and/or antimicrobial feature 32 to further prevent potential contamination thereof. In one example, as shown in FIG. 2, the feature 32 includes a sterilizable and/or antimicrobial coating 32′ is disposed on an exterior surface 34 of the main belt body 12. Although shown schematically in FIG. 2 as only covering a portion of the main belt body 12, it will be appreciated that the sterilizable and/or antimicrobial coating 32′ covers substantially the entire exterior surface 34 of the main band body 12 to facilitate sterilization and/or suppress microbial contamination. In another example, the sterilizable or antimicrobial feature 32 includes a sterilizable material or antimicrobial agent layer 32″ embedded within the main band body 12. The sterilizable and/or microbial feature 32 can include any suitable material, such as a heat-resistant coating to facilitate heat sterilization, a chemical-resistant layer to enable chemical sterilization, or an antimicrobial agent such as zeolite.

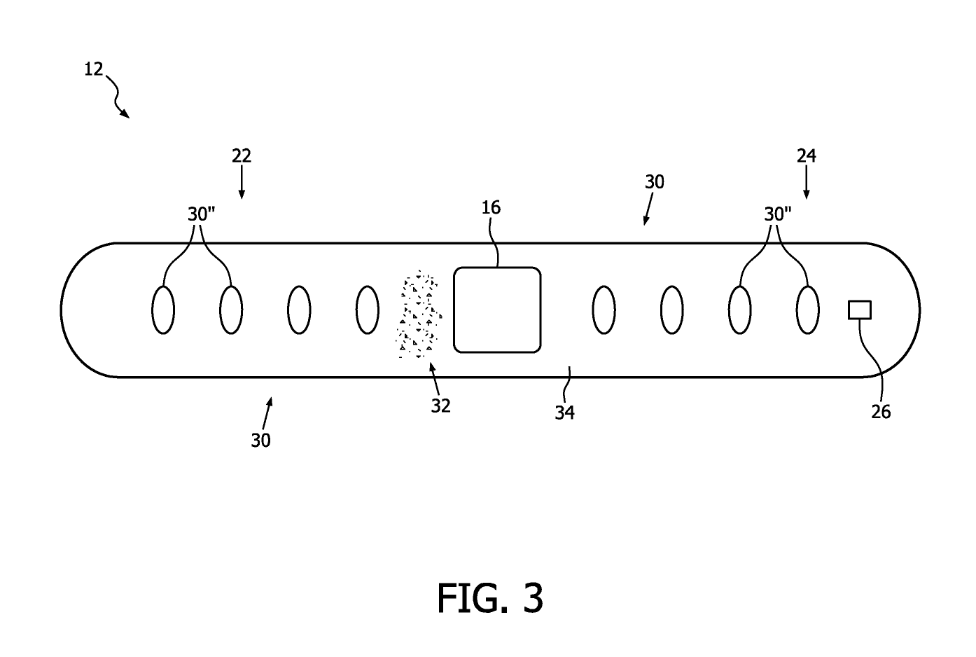

In some examples, the main band body 12 includes an integrated shield membrane 36 for sealing off a path between the medical subject and the main band body 12 from light and/or air during measurement (e.g., SpO₂ or temperature) by the medical monitoring device 14. In other examples, the main band body 12 can provide such a seal without the shield membrane 36 (i.e., solely with the main band body 12). FIG. 3 shows an alternative embodiment of the band 10. In this embodiment, the ferromagnetic layer 30′ has been replaced with one or more ferromagnetic rivet portions 30″. The ferromagnetic, e.g., stainless steel, rivets 30″ are exposed on, and are spaced along, a length of the main band body 12. As shown, the rivets 30″ have a generally elliptical shape; although other shapes are possible (e.g., circular, rectangular, square, and the like). The rivets 30″ are made of a magnetic material so as to engage the magnet 26. The rivets 30″ are spaced apart along the belt so as to advantageously provide different “sizes” of the main band body 12 so that the band 10 can fit around any sized patient (i.e., similar to the way that belt notches work).

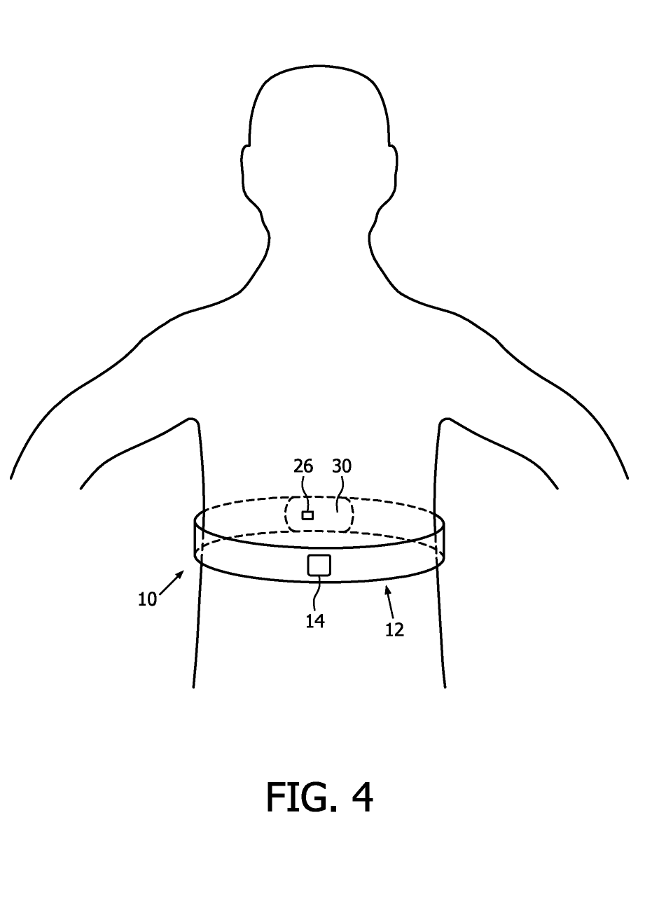

As shown in FIG. 4, the belt or band 10 is secured to the chest of the patient. To do so, the main belt body 12 is wrapped around a target tissue of the patient (the chest in this illustrative example) until portions of each of the first and second end portions 22 and 24 overlap each other. The magnet 26 then engages the ferromagnetic portion 30 (e.g., the ferromagnetic layer 30′ or the ferromagnetic rivets 30″) to secure the belt 10 to the patient. Advantageously, the belt 10 is configured for self-adjusting a tightness thereof around the patient. In one example, the belt 10 can be wrapped around the target tissue (e.g., the chest) to be slightly over-tight therearound. This tightness causes slippage of the magnet 26, thereby providing loosening of the belt 10 to a desired tightness (e.g., during sliding of the first end portion 22 along the magnet 26). Said another way, the strength of the magnetic coupling between the magnetic buckle 26 and the ferromagnetic material 30, 30′, 30″ serves as a limiter on the tightness of the buckled belt. In some embodiments, the belt 12 is elastic, which allows the belt to elastically expand to a desired fit tightness. Elasticity of the belt 12 also advantageously accommodates chest expansion and contraction during respiration. Having a controlled tightness of the magnetically cinched belt 10 facilitates accurate respiratory measurements.

To complete setup for the respiration measurement, the medical monitoring device 14 is secured to the belt 10, e.g., at the designated device mounting location 16 if such is defined, or anywhere along the belt 10 if, for example, the belt is embedded with magnetic material 30 along its entire length and the monitoring device 14 includes the illustrative device mounting magnet 20. In the illustrative example of an accelerometer-based respiratory monitoring device 14, the monitoring device 14 advantageously does not need to directly contact the patient, so long as the belt 10 secured around the patient moves (for example, expands and contracts in the case of an elastic belt) with the respiratory chest motion. The disclosed approach is also suitable for other types of monitors for example, an optically-based SpO₂ (oximeter) monitor may be similarly mounted with its light source illuminating the patient's skin through an aperture opening in the belt 10. (In this case, the oximeter monitor would need to be mounted at the illustrative specific device mounting position 16 so as to align with such an aperture). A sensor employing physical contact with the patient's skin is also contemplated, in which case the contact would again be suitably through an aperture opening in the belt 10.

Additionally, re-positioning of the belt, or of the monitoring device on the belt, is straightforward. In another example, the magnet 26 may become disengaged from the ferromagnetic portion 30 (e.g., the ferromagnetic layer 30′ or the ferromagnetic rivets 30″). The magnet 26 can be disengaged from the ferromagnetic portion 30 by being too tight, too loose, the patient bumping into an object while wearing the band 10, and the like. The belt 10 can be adjusted by moving the second end portion 24 (i.e., the magnet 26) towards or away from the magnet 26 to tighten or loosen the belt 10 accordingly. It will be appreciated that the medical monitoring device 14 can be secured to the device-mounting portion 16 before or after the belt 10 is secured to the patient.

The invention has been described with reference to the preferred embodiments. Modifications and alterations may occur to others upon reading and understanding the preceding detailed description. It is intended that the invention be constructed as including all such modifications and alterations as they come within the scope of the appended claims or the equivalents thereof

1. A band for positioning a medical monitoring device on a portion of a target portion of a medical subject, the band comprising:
a flexible main band body comprising at least one of at least one plastic layer, a textile layer, and a flexible rubber layer;
at least one ferromagnetic portion disposed on or in at least a first end portion of the flexible main band body; and
at least one magnet disposed on or in a second end portion .(of the flexible main band body opposite from the first end portion; and
wherein the flexible main band body is sized to wrap around the target portion of the medical subject with the first and second end portions overlapping such that the at least one magnet and the at least one ferromagnetic portion magnetically couple to secure the flexible main band body wrapped around the target portion of the medical subject.

2. The band according to claim 1, wherein the flexible main band body comprises at least one polyurethane layer.

3. The band according to claim 1, wherein the ferromagnetic portion includes a ferromagnetic material layer embedded in the flexible main band body at least in the first end portion.

4. The band according to claim 1, wherein the flexible main band body includes at least one silicone layer, the ferromagnetic portion making the belt conductive, thereby making the main band body non-static.

5. The band according to claim 1, further including a device-mounting portion disposed on or formed in the flexible main band body and configured to receive the medical monitoring device, the device mounting portion including a breathable portion.

6. The band according to claim 1, wherein the band is configured for self-adjusting tightness by the magnetic coupling of the at least one magnet and the ferromagnetic portion having slippage effective to release any over-tightness of the wrapping of the flexible main band body around the target portion of the medical subject.

7. The band according to claim 1, wherein the at least one magnet include an electromagnetic portion, the ferromagnetic portion having slippage controlled by adjusting a voltage applied to the flexible main band body to release any over-tightness of the wrapping of the flexible main band body around the target portion of the medical subject, the electromagnet including a release functionality based on the medical monitoring accelerometer device and an emergency release algorithm input.

8. The band according to claim 1, wherein at least one ferromagnetic portion includes ferromagnetic rivets disposed along the first end portion of the flexible main band body.

9. The band according to claim 1, further including a sterilizable or antimicrobial coating disposed on an exterior surface of the main band body or embedded within the main band body.

10. The band according to claim 1, wherein the main band body is free of seams, thereby preventing contamination thereof.

11. The band according to claim 1, further including a shield membrane for sealing off a path between the target portion and the main band body from light and/or air.

12. A respiratory monitor comprising:
a band as set forth in claim 1 sized to wrap around the chest of a medical subject; and
a medical monitoring device comprising an accelerometer-based respiratory monitoring device including a device mounting magnet effective to secure the medical monitoring device to the main band body via magnetic coupling of the device magnet with the at least one ferromagnetic portion.

13-21. (canceled)

22. A respiratory monitoring method comprising:
wrapping a belt around the chest of a subject;
magnetically buckling the belt wrapped around the chest of a subject by magnetically coupling a buckle magnet disposed at one end of the belt with ferromagnetic material disposed in or on a portion of the wrapped belt overlapping the buckle magnet; and
monitoring patient respiration using a respiratory monitor attached to the belt.

23. The respiratory monitoring method of claim 22 further comprising:
attaching the respiratory monitor to the belt after magnetically buckling the belt wrapped around the chest of a subject.
